(19) United States
(12) Patent Application Publication
(10) Pub. No.: US 2013/0018273 A1
(43) Pub. Date: Jan. 17, 2013

(54) SYSTEMS AND METHODS FOR ANALYSIS OF FETAL HEART RATE DATA

(71) Applicants: Lianna M. Hall, Lowell, MA (US); Michael House, Belmont, MA (US); Matthias Muenzer, Brookline, MA (US)

(72) Inventors: Lianna M. Hall, Lowell, MA (US); Michael House, Belmont, MA (US); Matthias Muenzer, Brookline, MA (US)

(73) Assignee: TUFTS UNIVERSITY, Medford, MA (US)

(21) Appl. No.: 13/548,392

(22) Filed: Jul. 13, 2012

Related U.S. Application Data
(60) Provisional application No. 61/508,239, filed on Jul. 15, 2011.

(51) Int. Cl. A61B 5/0444

(52) U.S. Cl. 600/511

(57) ABSTRACT

The present invention relates to systems and methods for analyzing fetal heart rate data. In particular, the present invention provides electronic rulers for use in analyzing fetal heart rate data.

### CROSS-REFERENCE TO RELATED APPLICATION

The present application claims priority to U.S. Provisional Patent Application Ser. No. 61/508,239 filed Jul. 15, 2011, which is hereby incorporated by reference in its entirety.

### FIELD OF THE INVENTION

The present invention relates to systems and methods for analyzing fetal heart rate data. In particular, the present invention provides electronic rulers for use in analyzing fetal heart rate data.

### BACKGROUND OF THE INVENTION

Electronic fetal monitoring (EFM) devices are highly utilized in labor and delivery departments, but the accuracy and reliability of their use by clinicians are questionable. Obstetric practitioners perceive fetal heart rate (FHR) interpretation accuracy differences when viewing FHR tracings on paper versus electronic displays.

FHR monitoring is a technique that can be performed manually by intermittent auscultation of the fetal heart rate (e.g., listening to the FHR by using a stethoscope), or electronically by using an electronic fetal monitoring (EFM) device.

The first step is to describe the individual features of the fetal heart rate and contractions. The second step is to interpret the combination of these features. The third step is to assess the fetal condition by considering the overall FHR interpretation in conjunction with additional clinical factors. The last step is to decide the management course given the fetal assessment. Studies consistently report results in which poor reliability are shown among physicians, residents, nurses, and midwives, but offer no root cause for why reliability results are low across the fetal monitoring decision-making steps.

Additional methods to analyze EFM data are needed.

### SUMMARY OF THE INVENTION

The present invention relates to systems and methods for analyzing fetal heart rate data. In particular, the present invention provides electronic rulers for use in analyzing fetal heart rate data.

Embodiments of the present invention provide software, systems and methods that overcome many of the limitation of existing systems for monitoring FHR information. For example, in some embodiments, the present invention provides systems and methods comprising: A processor and software comprising instructions for analyzing electronic fetal heart rate (FHR) data, generating an assessment of FHR information; and displaying the assessment. In some embodiments, the instructions further comprising instructions for overlaying an electronic ruler on an electronic trace of FHR data and using the ruler to generate the assessment. In some embodiments, the FHR information is, for example, FHR variability, deceleration events and acceleration events. In some embodiments, the electronic ruler comprises a plurality of horizontal bands (e.g., for indicating levels of variability of FHR). In some embodiments, the electronic ruler comprises a plurality of boxes (e.g., indicating time periods for measuring acceleration or deceleration events). In some embodiments, the ruler can be scaled or moved by an operator. In some embodiments, the assessment comprises determination of the presence of absence of fetal distress. In some embodiments, the software further comprises instructions for recommending a treatment course of action based on the presence or absence of fetal distress (e.g., continuing with labor, providing treatment to speed labor or providing a cesarean section). In some embodiments, the system further comprises a device for measurement and collection of FHR data.

### DESCRIPTION OF THE FIGURES

FIG. 1 shows a schematic of a FHR interpretation interactive ruler of embodiments of the present invention.

FIG. 2 shows an example of a FHR tracing electronic display.

FIG. 3 shows an interactive ruler of embodiments of the present invention displayed with a FHR tracing.

### DEFINITIONS

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human or non-human mammal subject.

As used herein the terms “processor,” “digital signal processor,” “DSP,” “central processing unit” or “CPU” are used interchangeably and refer to a device that is able to read a computer program (e.g., software) and perform a set of steps according to the program.

### DETAILED DESCRIPTION OF THE INVENTION

The present invention relates to systems and methods for analyzing fetal heart rate data. In particular, the present invention provides electronic rulers for use in analyzing fetal heart rate data.

Embodiments of the present invention solve multiple problems of prior methods of analyzing FHR data. The problems addressed by the invention fall into two categories: visual perception and cognition in the course of clinicians performing FHR interpretation. The visual perception of baseline FHR variability jaggedness is challenging when a clinician discriminates a noisy FHR tracing that is drawn on a static background reference grid. The visual perception is further challenging for determining jaggedness in the 5-10 bpm range, which is clinically an important range to identify. Additionally, the bpm grid markers on commercial EFM displays are displayed infrequently, which then cause users to have to scan and locate the markers to measure the jaggedness. Clinicians must perform mental math and may often estimate the jaggedness for expediency. Also, the spatial separation of the FHR tracing from the contraction data is large enough to make an assessment of deceleration type challenging and error prone.

The cognition issues of baseline FHR variability interpretation consist of the need both to view changes in baseline FHR variability over a long time period and to memorize the FHR interpretation guidelines. In order to view FHR changes over time, a clinician must scroll the display and view ˜8-10 minutes of a tracing at a time, which causes additional cognitive workload because the FHR tracing assessment must be retained in memory. The cut-off used for minimal (5 bpm) and moderate (6 bpm) FHR variability may vary from clinician to clinician and introduces lower inter-rater reliability. In practice, the FHR guidelines consist of knowledge in the head instead of knowledge in the world, which can lead to their misrepresentation.

No interactive visual aids are currently in place on electronic fetal monitoring systems in either commercial or research settings. Obstetric clinicians must perform visual fetal heart rate (FHR) interpretation using monitors that do not include interactive decision-support tools. Commercial and research EFM systems may automatically calculate fetal heart rate features and generate alarms based on these calculations. Such automation may or may not be considered in the course of EFM decision-making based on a clinician's perception of the automation's reliability.

The OBTraceVue system (Philips Healthcare, Andover, MA) presents a tabular flow sheet that summarizes the system's FHR variability calculation results. The CALM Patterns™ product (LMS Medical, New York. N.Y.) determines a baseline FHR, which excludes accelerations and decelerations, and then calculates FHR variability as the range of FHR values that fall within two standard deviations of the baseline FHR (Elliott, Warrick, Graham, & Hamilton, 2009, Am J Obstet Gynecol, 201). The Sonicaid: System 8000 system calculates both short- and long-term variability (Dawes, Moulden, & Redman, J Perinat Med, 19, 47-51 1991), as do the SisPorto® system (Ayres-de-Campos, Costa-Santos, & Bernardes, Eur J Obstet Gynecol Reprod Biol, 118, 52-60 2005) and the 2CTG2 system (Magenes, Signorini, Ferrario, & Lunghi, IFMBE Proceedings 16, 781-784 2007).

Embodiments of the present invention provide the advantage of aiding fetal assessment in ways that keep the decision-making in the hands of the obstetric practitioners. The interactive ruler of embodiments of the present invention aids FHR interpretation by 1) reducing visual perception problems, 2) reducing the need to calculate or measure heart rate features, 3) aiding memory for FHR interpretation over an extending time period, 4) and aiding memory of FHR interpretation guidelines by embedding the definitions.

Exemplary, non-limiting embodiments of the present invention are described below.

### I. Ruler

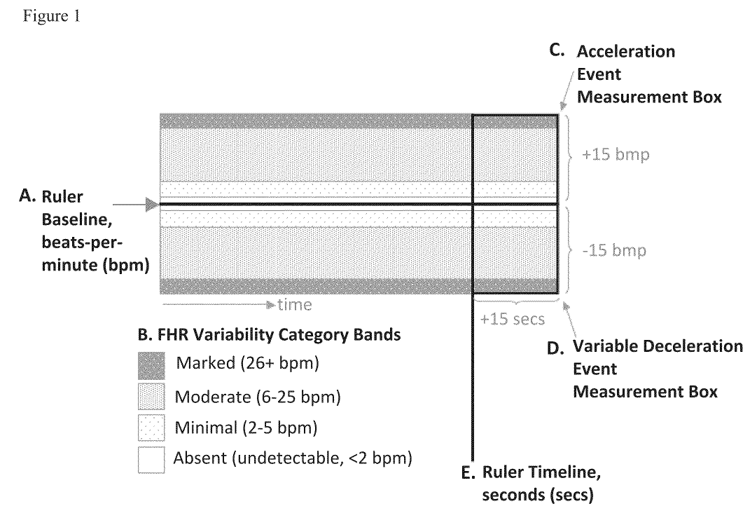

In some embodiments, the present invention provides an electronic ruler or measurment software component that can be integrated on existing electronic fetal monitor (EFM) displays. In some embodiments, the ruler design includes features that embed the 2008 FHR interpretation guidelines (Macones, G. A., Hankins, G., Spong, C. Y., Hauth, J., & Moore, T. (2008). Obstetrics & Gynecology, 112(3), 661-666) for interpreting baseline FHR, baseline FHR variability, an acceleration event, and a deceleration event. In some embodiments, the ruler design also includes a feature to aid the visual discrimination of a deceleration type (e.g., early, late, variable). Exemplary components of the ruler and support for the FHR interpretation of each FHR feature are described below and map to the design annotations provided in FIG. 1.

### Electronic Ruler Design

A. Ruler Baseline: In some embodiments, the baseline FHR is obtained by moving the Ruler Baseline to a location that visually appears as the average FHR within stable (mostly horizontal) FHR tracing segments.
B. FHR Variability Category Bands: In some embodiments, four separate horizontal bands, each visually distinct (e.g., color, pattern, shading, etc.), are vertically sized to represent each of the four baseline FHR variability categories (absent, minimal, moderate, marked). The bands are vertically centered on the Ruler Baseline so that a user can assess the amount of vertical, beat-to-beat jaggedness (oscillations) around the baseline FHR to categorize baseline FHR variability without having to explicitly perform a mathematical measurement.
C. Acceleration Event Measurement Box: An acceleration event is defined, for example, as an FHR that increases by 15 bpm for a minimum of 15 seconds (for a non-premature fetus). In some embodiments, such an event is measured by using the boundaries of an acceleration box.
D. Variable Deceleration Event Measurement Box: A variable deceleration, which is one of three types of deceleration events, is defined, for example, as an FHR that decreases by 15 bpm for a minimum of 15 seconds. In some embodiments, such an event is measured by using the boundaries of a deceleration box.
E. Ruler Timeline: A deceleration type is determined by evaluating a deceleration in the context of a contraction peak. The Ruler Timeline is a vertical line that displays at a point in time, and is drawn from within the FHR tracing display area on down into the uterine contraction display area. The user can place the Timeline at the lowest point of a deceleration (called the nadir), and assess the relationship of the line to the peak of a contraction. If the deceleration nadir occurs at the same as the contraction peak then the deceleration is considered early. If the deceleration nadir occurs after the contraction peak then the deceleration is considered late.

Additional design elements (not shown in FIG. 1) include, for example, support for representing “abrupt” FHR changes (<30 seconds) and “gradual” FHR changes (>30 seconds).

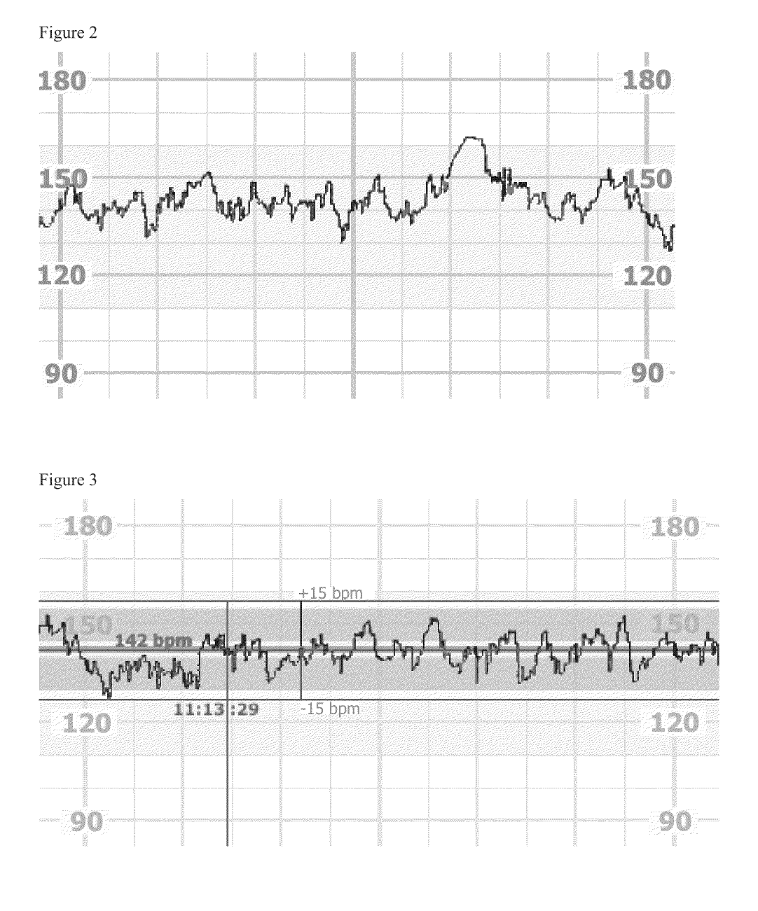

Commercial EFM displays consists of a FHR tracing displayed in the top section, an example of which is shown in FIG. 2, and a uterine contraction area in the bottom section (not shown). The FHR and contraction data are displayed from left to right over time, and a horizontal scrollbar enables obstetric clinicians to view hours of prior FHRs and contractions. An example of a Ruler component drawn into the FHR tracing area of an EFM display is provided in FIG. 3.

### Ruler Behavior

In some embodiments, the ruler supports a core set of features, which include, for example, the following:
1. In some embodiment, the Ruler is moved using a computer mouse, a touch-screen or additional input devices.
   a. The vertical movement allows the placement of the Ruler Baseline at a particular fetal heart rate, and allows the Baseline to be moved higher and lower increments of 1 bpm.
   b. The horizontal movement allows the placement of the Ruler Timeline at a particular time (HH:MM:SS), and allows the Timeline to be moved forward (to the right) and backward (to the left) in increments of 1 second and 1 minute.
   c. The Ruler remains located in the same vertical position when a FHR tracing window is scrolled back and forward in time.
2. In some embodiments, the Ruler scales to accommodate any change in its corresponding FHR tracing display scale, such as for a zoom in or zoom out capability.
3. In some embodiments, a capability to dynamically (interactively) change the width of the Ruler is provided, along with a dynamic read-out of the corresponding time duration.
4. In some embodiments, a capability to dynamically (interactively) change the height of the Ruler is provided, along with a dynamic read-out of the corresponding beats-per-minute amplitude.
5. In some embodiments, a capability to dynamically rotate the Ruler is provided.
6. In some embodiments, a capability to print a FHR tracing display with the Ruler is provided.
7. In some embodiments, a capability to fully obscure the underlying static grid, and include the grid within the Ruler is provided.

### Ruler Display Features

Additional features for setting the visual aspects of the ruler include, but are not limited to, the following:
1. Set the color (hue), intensity, and transparency of the Ruler Baseline, though, the Baseline should always be visible.
2. View/hide individual Ruler Bands (e.g., variability categories).
3. Set the color (hue), intensity, and transparency of each Ruler Band.
4. View/hide Ruler-specific grid markings (current bpm, current time, +and −15 bpm), along with acceleration and deceleration Event Measurement Boxes.
5. Pre-configure an intrapartum Ruler setting that contains the Absent Band and Ruler Timeline.
6. Pre-configure an antepartum Ruler setting that contains all features of the Ruler.
7. Allow a user to preset the width of the Ruler to full-display (the default), or to a particular time increment (e.g., 2 minutes wide).

In some embodiments, four colored bands are displayed for each of the four FHR variability categories, where each band is centered on the baseline such that the height from top of a band above the baseline to the bottom of the band below the baseline represents the full amplitude range for a particular FHR variability category. To interpret variability, one places the ruler's baseline on the FHR baseline using a mouse, and then determines which band the variability's jaggedness fits within.

In some embodiments, absent variability is drawn as a red band that is just under two bpms. The NICHD guidelines describe absent variability as having an “undetectable” amplitude range. A band of red that is just under 2 bpm is used to quantify absent variability, in a manner similar to how current standards define the absent bandwidth range. Minimal variability is drawn as a yellow band that ranges from 2-5 bpm. Moderate variability is drawn as a green band that ranges from 6-25 bpms. Marked variability begins in the area immediately outside of the moderate variability area, which starts at 26 bpms, and inherently extends beyond the total ruler height of 30 bpm. The size of the bands are designed to depict comparisons and context to enable FHR features to be detected within the scope of the eyespan.

In some embodiments, the ruler horizontally fills the entire FHR tracing display area, and is 30 bpm in height, which provides two different 15 bpm vertical measurement areas, from the baseline up and from the baseline down. This design enables an easy detection of accelerations and variable decelerations across the visible FHR tracing. In addition to the horizontal ruler bands, in some embodiments a vertical timeline is drawn, which is designed to extend down into the uterine contraction area so that clinicians can more easily determine the type of deceleration by directly comparing its starting and lowest locations with the starting and peak locations of a contraction.

In some embodiments, two boxes 15 seconds wide by 15 bpm tall are drawn along the timeline and centered around the baseline to support the interpretation of accelerations and decelerations that are smaller in size or on a boundary. The top box is labeled “+15 bpm” for accelerations and the bottom box is labeled “−15 bpm” for variable decelerations. The timeline is moved back (to the left) and forward (to the right) in time with a mouse, and can be located at what one considers the start of an acceleration or deceleration so the FHR feature can be compared to the bounding box.

In some embodiments, a real-time update of the timeline's location is provided in MM:SS format, along with a real-time update of the ruler's baseline, near the appropriate reference points on the ruler.

FHR variability is viewed over a several minute time period, and is interrupted by accelerations, decelerations, and areas of the FHR that are overall unstable. Color, in combination with size and intensity differences, can help those users who are colorblind. In some embodiments, the ruler's bands are colored to support the relative seriousness of each FHR variability category to be communicated, with red for danger (e.g., absent variability), yellow for warning (e.g., minimal and marked variability), and green for safety (e.g., moderate variability), although other color or labeling schemes may be utilized.

In some embodiments, ruler labels include marking acceleration and deceleration areas, grid tick marks and labels that allow amplitude values to be calculated.

### II. Systems

In some embodiments, the present invention provides systems comprising the FHR analysis ruler of embodiments of the present invention. In some embodiments, a system is provided comprising FHR monitoring devices and systems, fetal heart rate analysis ruler software, along with additional software (e.g., software for data collection, data analysis, user interfaces, etc.) and computer systems for collecting data, running software, and displaying results to a user.

In some embodiments, software for FHR rulers is incorporated into existing FHR monitoring systems. In some embodiments, commercially available system are utilized (e.g., OBTraceVue (central monitoring by Philips Medical Systems), QS® Perinatal (GE Healthcare), CALM Patterns™ (LMS Medical Systems), and Sonicaid: System 8000 (Huntleigh Healthcare)).

III. Uses

The FHR analysis ruler of embodiments of the present invention finds use in any number of applications. For example, in some embodiments, the ruler is utilized in assessing fetal distress or determining normal fetal heart rate. In some embodiments, the ruler is utilized during labor and delivery to assess fetal condition and determine a treatment course of action (e.g., allowing labor to continue or intervening with techniques to speed labor and/or delivery (e.g., use of cesarean section)). In other embodiments, the ruler is utilized during routine prenatal assessment or during non-stress tests (NSTs), which are conducted using EFM throughout a pregnancy.

In some embodiments, a computer-based analysis program is used to translate the raw data generated by the ruler (e.g., presence or absence of abnormal FHR) into data of predictive value for the clinician (e.g., probability of fetal distress). The clinician (e.g., midwife, physician, etc.) can access the predictive data using any suitable means. Thus, in some preferred embodiments, the present invention provides the further benefit that the clinician need not understand the raw data of the FHR data. The data is presented directly to the clinician in its most useful form. The clinician is then able to immediately utilize the information in order to optimize the care of the subject.

The present invention contemplates any method capable of receiving, processing, and transmitting the information to and from medical personal and subject. For example, in some embodiments of the present invention, a subject is monitored (e.g., using EFM) and submitted to a remote service for analysis using the systems and methods of embodiments of the present invention. In this way, data obtained in remote areas or developing nations can be analyzed by a service in another location. For example, rather than providing raw data, the prepared format may represent a risk assessment for various treatment options the clinician may use or as recommendations for particular treatment options. The data may be displayed to the clinician by any suitable method. For example, in some embodiments, the analysis service or computer system generates a report that can be printed for the clinician (e.g., at the point of care) or displayed to the clinician on a computer monitor.

The present invention provides a unique system for specifically monitoring and tracking empirical results. For example, the success or failure of particular treatment options, selected using the FHR ruler and systems of the present invention, can be compiled in a database to empirically determine more accurate systems for generating and reporting suggested treatments and interventions. Using this monitoring and tracking system, the systems and methods of the present invention continuously evolve to improve results. The use of such systems by medical facilities improves the standard of care, while creating more efficiency and predictability in the management of medical businesses. The present invention thus provides a coordinated, timely, and cost effective system for obtaining, analyzing, and distributing life-saving information.

### EXPERIMENTAL

The following examples are provided in order to demonstrate and further illustrate certain preferred embodiments and aspects of the present invention and are not to be construed as limiting the scope thereof.

### Example 1

### FHR Ruler

Rulers were developed with the LabVIEW 2009 graphical programming language on a MacBook Air laptop.

The standard FHR tracing display included a static, scrollable 10-minute FHR tracing area. Given the static nature of the display, and for programming convenience, the 1-minute time labels were built into the surrounding area and not inside the FHR tracing display area. The interactive variability ruler was built using a mouse interaction.

The standard FHR display area is 1800 pixels wide by 420 pixels tall, for which a 10-second wide by 10-bpm tall ‘FHR block’ physically occupies 0.825 cm by 0.55 cm, respectively, on the electronic display. As such, the resulting aspect ratio is 0.667, which matches the accordion paper's aspect ratio. The FHR display area is based on a LabVIEW Picture control into which different display elements are drawn in a sequence to achieve particular visual effects.

The background grid is drawn first, and then the ruler is drawn on top of the grid with transparent colors so that the underlying grid is still visible. The FHR tracing is drawn last, which allows it to be viewed without being obscured. The FHR tracing is composed of a portable network graphic (PNG-24) image file that allows LabView to display the image as a transparent layer so that the underlying ruler and background grid are visible. Some underlying components were built in LabVIEW to convert data between clinical units (bpm and time in seconds) and display pixel units, and to generate the background display.

The experimental apparatus consisted of support to proceed to the next tracing and to control the ruler, and it was built to surround the FHR tracing display area. To support mouse interactions with the ruler, two vertical sliders were provided on the left- and right-hand sides of the FHR display area. The full ruler is moved up or down in three different ways, 1) dragging the prototype's sliders, 2) clicking on the 1-bpm increment buttons, or 3) clicking directly at a location within the FHR display area to position the ruler's baseline. The current baseline text field and sliders are all synchronized regardless of the mechanism used to change the baseline. A slider was provided below the FHR display area to move the time lines back (to the left) and forward (to the right) in time.

The total number of tracings available for a user to view was provided below the FHR display area, along with the current tracing number and a button to allow one to proceed to the next tracing. Upon starting the prototype, a spreadsheet file containing FHR tracing image names is selected by the researcher, the details of which are used to establish the specific tracings to view and the order in which they are displayed.

The ruler was viewed on a Samsung SyncMaster 2033 computer monitor, with a screen resolution of 1600×900 pixels. The monitor has a viewable display area of 44.2 cm wide by 24.8 cm tall. At this monitor size and the aforementioned display scale, approximately eight minutes of an FHR tracing was available for viewing at a glance, which is similar to that of commercial EFM displays. The mouse that was used in conjunction with the Samsung monitor was a wireless Apple Magic Mouse.

The digital form of the paper-based FHR tracings was generated using a multi-step process, where the end goal was to have an FHR tracing saved in an image file that could be imported into LabVIEW in a way that would allow a reference grid to be visible underneath the FHR tracing. First, each tracing was professionally scanned using a high-resolution scanner, and was saved in a tagged image file format (tiff) at a resolution of 600 dots-per-inch (dpi). Next, each tiff image was individually loaded into Adobe Photoshop, in which it was additionally processed. The Photoshop image processing steps included: cropping to an area that includes 10 minutes wide by 210 bpm tall (for the 30-240 bpm range), 2) fine-tuned rotating, as needed, 3) removing the background grid lines and labels so that only the FHR tracing remained, 4) scaling to a resolution of 72 pixels/inch to reduce the resulting file size so the tracing image files would load into the prototype quickly, 5) saving the image as a portable network graphics (png-24) format with transparency on. The final png-24 image file has pixel dimensions of 1800 pixels wide by 420 pixels tall so that it fits the dimensions of the FHR display area and has no distortions.

Thirty-three FHR tracings were placed into one of four groups by using the knowledge elicitation sessions' interpretation results. Each of three groups had ten tracings to support subsequent data collection during user testing, and one group had three tracings to support related training activities. The criteria used to select a tracing's group were defined as follows. Group 1 was for tracings with full FHR variability categorical agreement among the knowledge elicitation (KE) subjects. Group 2 was for tracings that were considered categorically borderline; in looking at the KE subjects' categories and the original computer algorithm's amplitude calculation, they bordered either absent and minimal or minimal and moderate categories. Group 3 was for tracings that had low categorical agreement among the KE subjects, and low categorical agreement between the gold standard and the subjects from a previous study. Specifically, Group 3 has tracings that were categorized as marked and sinusoidal by subjects in the previous study, but the gold standard has no tracings with these categories. Group 4 has three training tracings that had high agreement among the KE subjects.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

What is claimed is:

1. A system comprising:
A processor and software comprising instructions for analyzing electronic fetal heart rate (FHR) data, generating an assessment of FHR information; and displaying said assessment.

2. The system of claim 1, wherein said instructions further comprising instructions for overlaying an electronic ruler on an electronic trace of FHR data and using said ruler to generate said assessment.

3. The system of claim 1, wherein said FHR information is selected from the group consisting of FHR variability, deceleration events, and acceleration events.

4. The system of claim 2, wherein said electronic ruler comprises a plurality of horizontal bands.

5. The system of claim 4, wherein said bands indicate levels of variability of FHR.

6. The system of claim 2, wherein said electronic ruler comprises a plurality of boxes.

7. The system of claim 6, wherein said boxes indicate time periods for measuring acceleration or deceleration events.

8. The system of claim 2, wherein said ruler can be scaled or moved by an operator.

9. The system of claim 1, wherein said assessment comprises determination of the presence of absence of fetal distress.

10. The system of claim 9, wherein said software further comprises instructions for recommending a treatment course of action based on said presence or absence of fetal distress.

11. The system of claim 1, further comprising a device for measurement and collection of FHR data.

12. A method, comprising:
a) collecting electronic fetal heart rate (FHR) data;
b) analyzing and generating an assessment of FHR information with a processor and software; and
c) displaying said assessment.

13. The method of claim 12, wherein said analyzing comprises overlaying an electronic ruler on an electronic trace of FHR data and using said ruler to generate said assessment.

14. The method of claim 12, wherein said FHR information is selected from the group consisting of FHR variability, deceleration events, and acceleration events.

15. The method of claim 13, wherein said electronic ruler comprises a plurality of horizontal bands.

16. The method of claim 15, wherein said bands indicate levels of variability of FHR.

17. The method of claim 13, wherein said electronic ruler comprises a plurality of boxes.

18. The method of claim 17, wherein said boxes indicate time periods for measuring acceleration or deceleration events.

19. The method of claim 13, wherein said ruler can be scaled or moved by an operator.

20. The method of claim 12, wherein said assessment comprises determination of the presence of absence of fetal distress.

21. The method of claim 20, further comprising the step of recommending a treatment course of action based on said presence or absence of fetal distress.

22. The method of claim 12, further the step of collecting FHR data from a subject.
